(19) United States
(12) Patent Application Publication
(10) Pub. No.: US 2022/0015883 A1
(43) Pub. Date: Jan. 20, 2022

(54) ELECTRIC TOOTHBRUSH

(71) Applicant: SHENZHEN CONVL TECH CO., LTD., Guangdong, Shenzhen (CN)

(72) Inventor: Liuyun PAN, Shenzhen (CN)

(21) Appl. No.: 16/753,333

(22) PCT Filed: Aug. 6, 2019
(86) PCT No.: PCT/CN2019/099435

§ 371 Date: Apr. 2, 2020

(30) Foreign Application Priority Data
Apr. 9, 2019 (CN) 201920490374.4

(51) Int. Cl. A61C 17/20; A61C 17/34; A61C 17/22

(52) U.S. Cl. CPC A61C 17/20; A61C 17/224; A61C 17/222; A61C 17/3481

(57) ABSTRACT

An electric toothbrush includes a handle, a toothbrush head, and a charging base. One end of the handle is configured for outputting ultrasonic vibrations. The toothbrush head is configured for simultaneously wrapping upper and lower rows of teeth of a human body. The toothbrush head is configured to be disposed on the end of the handle and vibrates with the end of the handle to simultaneously clean the upper and lower rows of teeth of the human body. The charging base is disposed at the other end of the handle and charges the handle. And the charging base includes a sterilization unit for sterilizing the handle and the toothbrush head. The electric toothbrush provided by the present application is convenient to operate, and is configured for being charged through a charging base and being sterilized after being used, with complete functions and a wide application range.

### TECHNICAL FIELD

The present application relates to the technical field of daily necessities, and more particularly to an electric toothbrush.

### BACKGROUND

Electric toothbrushes are new products that have appeared in recent years, and have revolutionized the field of oral health in foreign countries. Although the application of electric toothbrushes in China has not yet been popularized, it has been predicted that electric toothbrushes will become an important member of public health products. According to surveys, electric toothbrushes are more scientific and effective than ordinary toothbrushes. It can remove dental plaque more thoroughly, and reduce oral diseases such as gingivitis, periodontal diseases and gum bleeding. And it is also a daily necessity popular in many countries in Europe and America.

At present, the electric toothbrushes used in the market only have a single vibration function, and it is needed to constantly change the position the toothbrush head by hand to clean different parts of the teeth, and the toothbrush head is wet after being used and is easy to breed bacteria.

### TECHNICAL PROBLEMS

The objective of the present application is to provide an electric toothbrush, which solves the technical problems in the prior art of electric toothbrushes that the position of the toothbrush head needs to be manually adjusted to clean teeth at different positions and the toothbrush head easily breeds bacteria.

### TECHNICAL SOLUTIONS

In order to achieve the above-said objective, the present application provides an electric toothbrush, comprising a handle, a toothbrush head, and a charging base, one end of the handle is configured for outputting ultrasonic vibrations, the toothbrush head is configured for simultaneously wrapping upper and lower rows of teeth of a human body, and the toothbrush head is configured to be disposed on the end of the handle and vibrates with the end of the handle to simultaneously clean the upper and lower rows of teeth of the human body; and the charging base is disposed at the other end of the handle and charges the handle, and the charging base comprises a sterilization unit for sterilizing the handle and the toothbrush head.

Further, the toothbrush head comprises:

a bristle holder, in a tooth shape adapted to a shape of the upper and lower rows of teeth of the human body, and the bristle holder has a first surface and a second surface opposite to each other;
first bristles, disposed on the first surface and configured for wrapping and cleaning the upper row of teeth of the human body; and
second bristles, disposed on the second surface and configured for wrapping and cleaning the lower row of teeth of the human body.

Further, the toothbrush head further comprises a transmitting frame, and the transmitting frame comprises:

a transmitting body, inserted in the bristle holder and configured for forming a connection with the end of the handle; and
a plurality of transmitting rods, distributed on the transmitting body and configured for dispersing the ultrasonic vibrations on the transmitting body to various parts of the bristle holder.

Further, the bristle holder comprises a middle portion, and a first side portion and a second side portion respectively disposed at both ends of the middle portion; the first side portion, the middle portion and the second side portion are sequentially connected to form a tooth-shaped bristle holder; and the transmitting body is disposed on the middle portion, a part of the transmitting rods are disposed on the first side portion and are spaced along the width direction of the first side portion, and another part of the transmitting rods are disposed on the second side portion and are spaced along the width direction of the second side portion.

Further, the handle comprises:

an enclosure;
a circuit board, disposed in the enclosure;
a battery, disposed in the enclosure and configured for supplying power to the circuit board; and
a vibration motor, disposed in the enclosure and electrically connected to the circuit board, wherein a motor shaft of the vibration motor passes through the enclosure to be connected to the transmitting body.

Further, the enclosure comprises a bottom case and an upper cover, and the bottom case is engaged with the upper cover and sealed by a sealing ring; a first through hole is provided at a junction of the bottom case and the upper cover, and a button configured for driving the vibration motor is disposed in the first through hole; and the sealing ring is integrally connected to the button.

Further, the handle further comprises blue light lamps electrically connected to the circuit board and capable of emitting blue light to whiten teeth, the blue light emitted by the blue light lamp shines on the bristle holder through the enclosure, and the bristle holder is made of a light guide material.

Further, the handle comprises two blue light lamps, and the two blue light lamps are symmetrically disposed on opposite two sides of the motor shaft, respectively.

Further, the charging base further comprises a drying unit configured for drying the handle and the toothbrush head; and the charging base has two first pins, the handle is provided with two second pins electrically connected to the circuit board, the second pins have jacks, and the first pins are inserted into the jacks to charge the battery.

Further, a first magnetic member is disposed on the charging base, a second magnetic member is provided on the enclosure, and the first magnetic member and the second magnetic member are attracted to each other, so that the first pins are inserted into the jacks.

### BENEFICIAL EFFECTS

The electric toothbrush provided by the present application has the beneficial effect that, compared with the prior art, the toothbrush head of the electric toothbrush of the present application can be configured to wrap the upper and lower rows of teeth of the human body at the same time, so that all teeth can be cleaned at one time without swinging the handle, with simple and practical operation. At the same time, the charging base is provided with a sterilization device, so that the charging base can sterilize the handle and the toothbrush head while charging, and prevent bacteria from breeding on the toothbrush head. The electric toothbrush of the present application has complete functions.

### BRIEF DESCRIPTION OF THE DRAWINGS

In order to explain the technical solutions in the embodiments of the present application more clearly, the drawings used in the embodiments or the description of the prior art will be briefly introduced below. Obviously, the drawings in the following description are only some embodiments of the present application, and for those of ordinary skill in the art, other drawings may be obtained based on these drawings without paying creative labor.

FIG. 1 is a schematic structural view of an electric toothbrush according to an embodiment of the present application;

FIG. 2 is a schematic structural view of a toothbrush head in FIG. 1;

FIG. 3 is a schematic sectional view of a handle and a toothbrush head in FIG. 1;

FIG. 4 is a schematic exploded view of a handle in FIG. 1;

FIG. 5 is a schematic structural view of a handle in FIG. 1 after removing the enclosure;

FIG. 6 is a schematic structural view of a bottom case in FIG. 4;

FIG. 7 is a schematic structural view of the charging base in FIG. 1.

Reference numerals in the drawings are as follows:

1—charging base; 2—handle; 3—toothbrush head; 11—sterilization unit; 12—drying unit; 13—first pin; 14—first magnetic member; 15—second magnetic slot; 21—enclosure; 22—circuit board; 23—battery; 24—vibration motor; 25—control button; 26—button; 27—motor sleeve; 28—blue light lamp; 29—light-transmitting cover; 31—bristle holder 32—first bristle; 33—second bristle; 34—transmitting frame; 210—installation cavity; 211—bottom case; 212—upper cover; 213—sealing ring; 214—first through hole; 221—second pin; 241—motor shaft; 291—slot; 292—perforation; 311—first surface; 312—second surface; 313—middle portion; 314—first side portion; 315—second side portion; 320—dental cavity; 321—first set of bristles; 322—second set of bristles; 323 third set of bristles; 331—transmitting body; 332—transmitting rod; 2111—hook; 2112—second magnetic member; 2113—first magnetic slot; 2114—second through hole; 2121—engagement groove; 3311—shaft hole.

### EMBODIMENTS OF THE INVENTION

In order to make the technical problems, technical solutions and beneficial effects of the present application to be more clearly understood, the present application will be further described in detail with reference to the accompanying drawings and embodiments. It should be understood that the specific embodiments described herein are only used to explain the present application and are not intended to limit the present application.

It should be noted that when an element is referred to as being “fixed to” or “disposed on” another element, it may be directly or indirectly fixed to/disposed on the other element. When an element is referred to as being “connected to” another element, it can be directly or indirectly connected to the other element.

It should be understood that the terms “length”, “width”, “upper”, “lower”, “front”, “rear”, “left”, “right”, “vertical”, “horizontal”, “top”, “bottom”, “inner”, “outer”, etc. indicate orientations or positional relationships based on the orientations or positional relationships shown in the drawings, only for convenience of describing the present application and simplifying the description, rather than indicating or implying that the device or element referred to must have a particular orientation, or be constructed and operated in a particular orientation, therefore they should not be construed as limiting the present application.

In addition, the terms “first” and “second” are used for descriptive purposes only and cannot be understood as indicating or implying relative importance or implicitly indicating the number of technical features indicated. Therefore, the features defined as “first” and “second” may explicitly or implicitly include one or more of the features. In the description of the present application, the meaning of “a plurality of” is two or more, unless it is specifically defined otherwise.

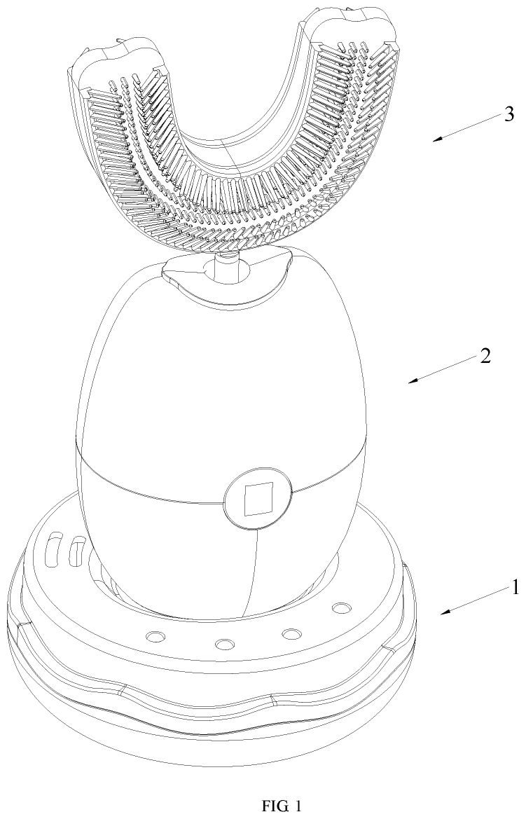

Referring to FIG. 1, the electric toothbrush provided by the present application will be described here. The electric toothbrush includes a handle 2, a toothbrush head 3 and a charging base 1. The handle 2 is used for hand holding. One end of the handle 2 can output ultrasonic vibrations. The toothbrush head 3 can simultaneously wrap the upper and lower rows of teeth of the human body. The toothbrush head 3 can be mounted on one end of the handle 2 and vibrates with the end of the handle 2 so as to simultaneously clean the upper and lower rows of teeth of the human body. The charging base 1 can be disposed on the other end of the handle 2 and charges the handle 2. The charging base 1 includes a sterilization unit 11 for sterilizing the handle 2 and the toothbrush head 3.

For the electric toothbrush according to the embodiments of the present application, when used for brushing teeth, the toothbrush head 3 is mounted on the handle 2, the handle 2 is held, the toothbrush head 3 is placed in the mouth, and the toothbrush head 3 simultaneously wraps the upper and lower rows of teeth of the human body, then the handle 2 is actuated so that one end of the handle 2 outputs ultrasonic vibrations, and drive the toothbrush head 3 to clean the teeth; and when the use is done, the other end of the handle 2 can be placed on the charging base 1, and the charging base 1 charges the handle 2 and sterilizes the handle 2 and the toothbrush head 3 through the sterilization unit 11 at the same time.

The toothbrush head 3 of the electric toothbrush provided by the present application can be configured to simultaneously wrap the upper and lower rows of teeth of the human body, so that all teeth can be cleaned at one time without swinging the handle 2, with simple and practical operation. At the same time, the charging base 1 is provided with a sterilization device 11, so that the charging base can sterilize the handle 2 and the toothbrush head 3 while charging, and prevent bacteria from breeding on the toothbrush head 3. The electric toothbrush of the present application has complete functions.

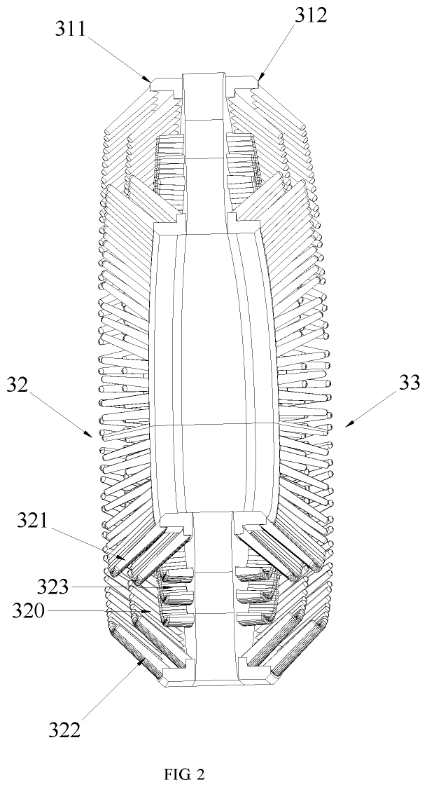

Further, referring to FIG. 2, as a specific embodiment of the electric toothbrush provided by the present application, the toothbrush head 3 includes a bristle holder 31, a plurality of first bristles 32, and a plurality of second bristle 33. The bristle holder 31 is tooth-shaped. The shape of the bristle holder 31 is adapted to the shape of the upper and lower rows of teeth of the human body. The bristle holder 31 has a first surface 311 and a second surface 312 opposite to each other in the left direction and right direction of FIG. 2. The first bristles 32 are provided on the first surface 311 and are configured for wrapping and cleaning the upper row of teeth of the human body, and the second bristles 33 are provided on the second surface 312 and configured for wrapping and cleaning the lower row of teeth. When in use, the bristle holder 31 is inserted horizontally into the mouth of the human body, so that the first bristles 32 wrap the upper row of teeth of the human body, and the second bristles 33 wrap the lower row of teeth of the human body, and then the handle is actuated to drive the bristle holder 31 to generate ultrasonic vibrations, thereby driving the first bristles 32 and the second bristles 33 to clean the teeth.

Specifically, referring to FIG. 2, the first bristles 32 include a first set of bristles 321, a second set of bristles 322, and a third set of bristles 323. The first set of bristles 321, the second set of bristles 322, and the third set of bristles 323 are all uniformly distributed along the longitudinal extending direction of the bristle holder 31, that is, the first set of bristles 321, the second set of bristles 322, and the third set of bristles 323 are all distributed along the tooth shape. The first set of bristles 321 and the second set of bristles 322 are symmetrically disposed on the inner and outer edges of the bristle holder 31. The first set of bristles 321 and the second set of bristles 322 are inclined to the middle, and the third set of bristles 323 is disposed between the first set of bristles 321 and the second set of bristles 322. The first set of bristles 321 is vertically inserted on the bristle holder 31, and the height of the first set of bristles 321 is less than the height of the second set of bristles 322 and the height of the third set of bristles 323. The first set of bristles 321, the second set of bristles 322, and the third set of bristles 323 are enclosed to form a dental cavity 320 for wrapping the teeth. When brushing teeth, the teeth are wrapped in the dental cavity 320, and at the same time, first set of bristles 321 wash the end of the teeth, and the second set of bristles 322 and the third set of bristles 323 wash both sides of the teeth respectively, thereby ensuring that all parts of the tooth are cleaned.

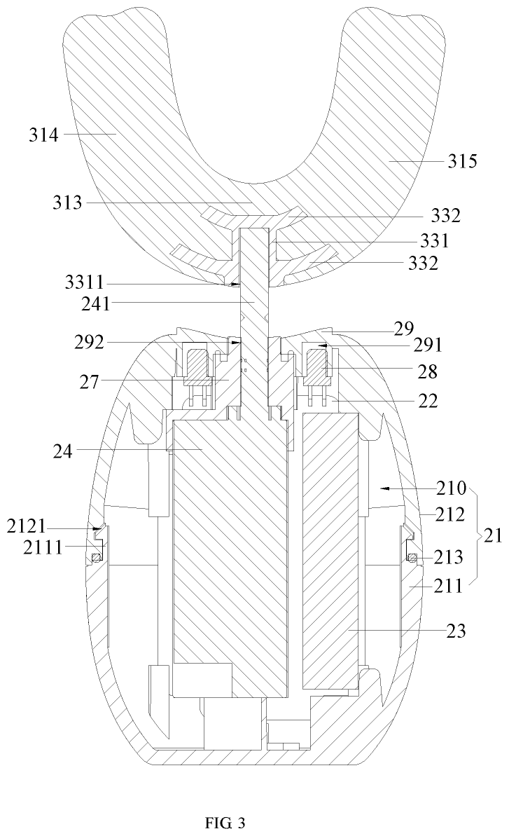

Further, referring to FIG. 3, as a specific embodiment of the electric toothbrush provided by the present application, the toothbrush head 3 further includes a transmitting frame 34 for transmitting and dispersing the ultrasonic vibrations at one end of the handle 2 to various parts of the toothbrush head 3. Specifically, the transmitting frame 34 includes a transmitting body 341 and four transmitting rods 342. The transmitting body 341 is inserted in the bristle holder 31. The transmitting body 341 is used to form a connection with one end of the handle 2 to receive ultrasonic vibrations on the handle 2. The transmitting rods 342 are distributed on the transmitting body 341, and each of the transmitting rods 342 is used to disperse the ultrasonic vibrations on the transmitting body 341 to various parts of the bristle holder 31. In this embodiment, the ultrasonic vibrations at one end of the handle 2 can be quickly and uniformly dispersed to various parts of the toothbrush head 3 through the setting of the transmitting frame 34, so that the toothbrush head 3 has a good cleaning effect on the teeth.

In this embodiment, the transmitting frame 34 includes four transmitting rods 342. It should be understood that, in other embodiments of the present application, according to the actual size and width of the bristle holder 31, the transmitting frame 34 may include two or six transmitting rods 342, which is not limited here.

Preferably, referring to FIG. 3, the bristle holder 31 includes a middle portion 313, a first side portion 314, and a second side portion 315. The first side portion 314 is connected to one end of the middle portion 313, and the second side portion 315 is connected to the other end of the middle portion 313. The middle portion 313 is used to correspond to the front teeth of the human body, and the first side portion 314 and the second side portion 315 are respectively used to correspond to the teeth on both sides of the human body. The first side portion 314, the middle portion 313, and the second side portion 315 are sequentially connected to from the tooth-shaped bristle holder 31. The transmitting body 341 is disposed on the middle portion 313. Two transmitting rods 342 are disposed on the first side portion 314 and are spaced along the width direction of the first side portion 314, and the length of the transmitting rod 342 close to the inner edge of the first side portion 314 is smaller than the length of the transmitting rod 342 close to the outer edge of the first side portion 314. The other two transmitting rods 342 are disposed on the second side portion 315 and spaced along the width direction of the second side portion 315, and the length of the transmitting rod 342 close to the inner edge of the second side portion 315 is smaller than the length of the transmitting rod 342 close to the outer edge of the second side portion 315.

Further, referring to FIGS. 3-5, as a specific embodiment of the electric toothbrush provided by the present application, the handle 2 includes an enclosure 21, a circuit board 22, a battery 23, and a vibration motor 24. The enclosure 21 presents a substantially drum shape. The circuit board 22 and the battery 23 are both disposed in the enclosure 21. The battery 23 is electrically connected to the circuit board 22 and supplies power to the circuit board 22. The vibration motor 24 is disposed in the enclosure 21 and is electrically connected to the circuit board 22. The vibration motor 24 can output ultrasonic vibrations under the driving of the circuit board 22. The motor shaft 241 of the vibration motor 24 passes through the enclosure 21 to connect to the transmitting body 341, so that the ultrasonic vibrations are transmitted to the toothbrush head 3 through the transmitting body 341.

Specifically, the transmitting body 341 defines a polygonal shaft hole 3311. The motor shaft 241 of the vibration motor 24 can be inserted into the shaft hole 3311 to form a connection with the transmitting body 341, and the motor shaft 241 of the vibration motor 24 forms an interference fitting connection with the shaft hole 3311.

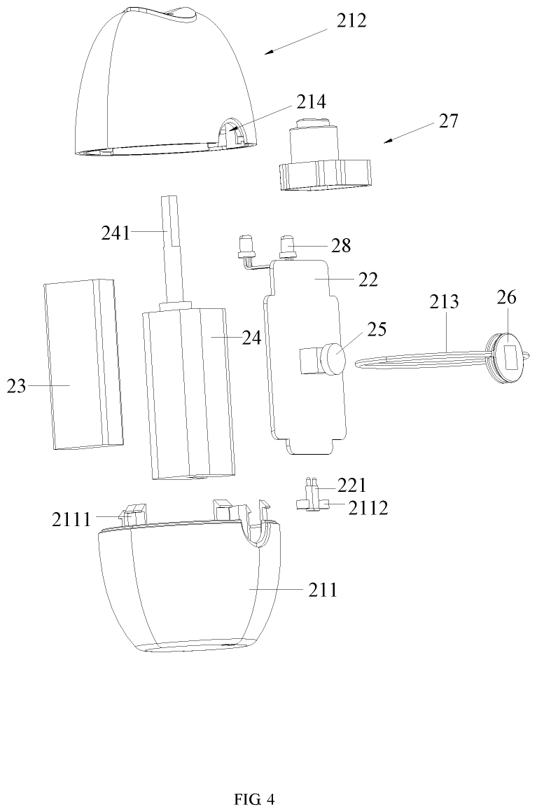

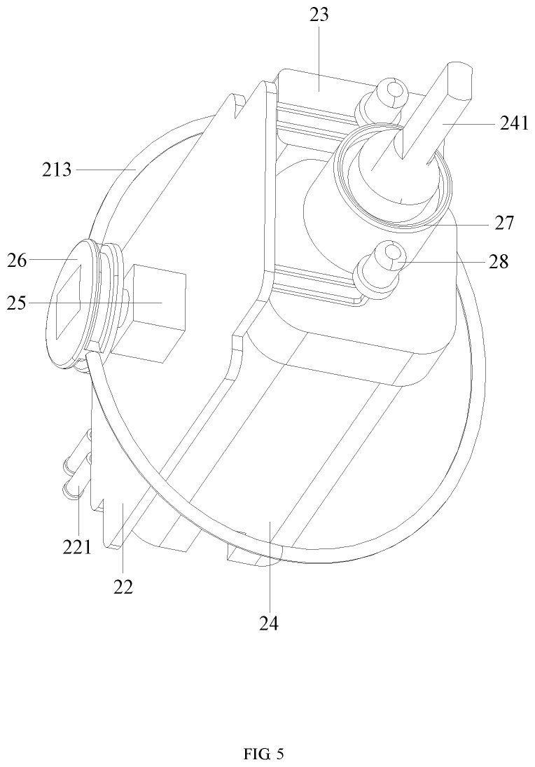

Referring to FIG. 3, the enclosure 21 presents a substantially drum shape. The enclosure 21 includes a bottom case 211 and an upper cover 212. The bottom case 211 is engaged with the upper cover 212 and sealed by a sealing ring 213. The bottom case 211 and the upper cover 212 are enclosed to form an installation cavity 210, and the circuit board 22, the battery 23, and the vibration motor 24 are all disposed in the installation cavity 210. Referring to FIG. 4 and FIG. 5, a control button 25 electrically connected to the circuit board 22 is disposed on the circuit board 22, and a first through hole 214 is defined at a junction of the bottom case 211 and the upper cover 212. A button 26 is disposed in the first through hole 214. The button 26 is in contact with the control button 25, and the button 26 is pressed to drive the control button 25 to activate the vibration motor 24. In this embodiment, the materials of the sealing ring 213 and the button 26 are both soft rubber materials, and the sealing ring 213 is integrally connected to the button 26. In this way, not only the button 26 and the sealing ring 213 can be formed by injection molding integrally using simple processing process, but also the sealing effect of the handle 2 is better.

In this embodiment, the bottom case 211 and the upper cover 212 are concavely and convexly matched and form a sealed connection through the sealing ring 213. The inner wall of the bottom case 211 is circumferentially provided with a plurality of hooks 2111. The inner wall of the upper cover 212 is circumferentially provided with a plurality of engagement grooves 2121. The hooks 2111 are engaged with the engagement grooves 2121 in a one-by-one correspondence, so that the bottom case 211 is more firmly connected to the upper cover 212. In actual installation, the sealing ring 213 may be firstly disposed on the bottom case 211, and then the upper cover 212 is covered on the sealing ring 213, and the upper cover 212 is pressed down to engage the hooks 2111 with the engagement grooves 2121.

Further, referring to FIG. 3, the vibration motor 24 and the battery 23 both extend along the axial direction of the enclosure 21. The bottom case 211 and the upper cover 212 are each provided with a plurality of mounting parts for positioning and mounting the battery 23 and the vibration motor 24. An end of the vibration motor 24 is provided with a motor sleeve 27, and the motor sleeve 27 is sleeved on the end of the vibration motor 24 and a part of the motor shaft 241. The motor sleeve 27 is mounted on the upper cover 212.

Further, referring to FIGS. 3-5, as a specific embodiment of the electric toothbrush provided by the present application, the handle 2 further includes a blue light lamp 28. The blue light lamp 28 is electrically connected to the circuit board 22 and can emit blue light so as to whiten teeth. The blue light emitted by the blue light lamp 28 shines on the bristle holder 31 through the enclosure 21, and the bristle holder 31 is made of a light guide material. In this way, the blue light emitted by the blue light lamp 28 can be transmitted through the light guide material and dispersed to each parts of the bristle holder 31, so that there is blue light to whiten the teeth at the same time of brushing teeth, and the teeth are cleaned and whitened, making the electric toothbrush multi-functional and have a wider application range.

Specifically, referring to FIG. 3, one end of the enclosure 21 defines a mounting opening (not shown in the figure), and a light-transmitting cover 29 is disposed at the mounting opening. The light-transmitting cover 29 is made of a transparent material. The inner side of the light-transmitting cover 29 defines slots 291, and the blue light lamps 28 are mounted in the slots 291 and are electrically connected with the circuit board 22 through wires. The blue light emitted by the blue light lamps 28 shines on the bristle holder 31 through the light-transmitting cover 29, and transmits and shines on the teeth through the bristle holder 31.

Further, the light-transmitting cover 29 defines a perforation 292 for the motor shaft 241 to pass through. One end of the motor sleeve 27 is wrapped around the end of the vibration motor 24, and the other end of the motor sleeve 27 extends to the perforation 292 and is mounted on the perforation 292.

Further, referring to FIGS. 3-5, as a specific embodiment of the electric toothbrush provided by the present application, the handle 2 includes two blue light lamps 28, and the two blue light lamps 28 are symmetrically disposed on opposite sides of the motor shaft 241, respectively. Specifically, the light-transmitting cover 29 defines two slots 291, and the two blue light lamps 28 are respectively mounted on the two slots 291. In this embodiment, two blue light lamps 28 are provided, thereby ensuring that blue light is transmitted to each part of the bristle holder 31. It can be understood that, in other embodiments of the present application, according to the actual shape and size of the bristle holder 31, the handle 2 may include three or more blue light lamps 28, which is not limited here.

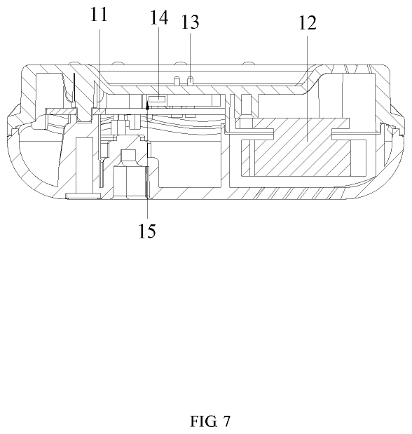

Further, referring to FIG. 7, as a specific embodiment of the electric toothbrush provided by the present application, the charging base 1 further includes a drying unit 12. The drying unit 12 is configured for drying the handle 2 and the toothbrush head 3. In this embodiment, the drying unit 12 is provided, so that the charging base 1 can dry the handle 2 and the toothbrush head 3 by the drying unit 12 while charging the battery 23, thereby preventing the breeding of bacteria.

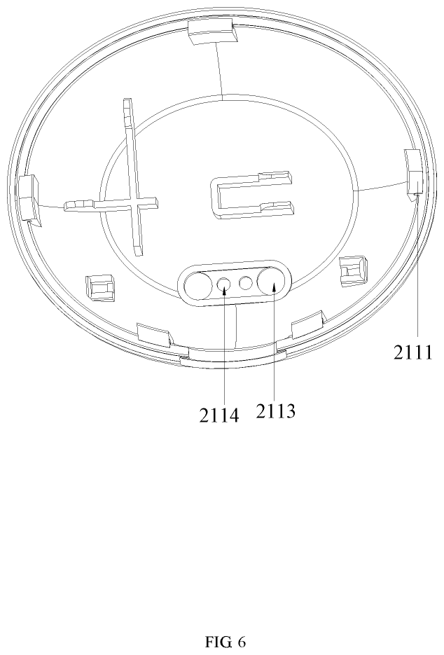

Preferably, in this embodiment, referring to FIGS. 4-7, the charging base 1 has two first pins 13, the handle 2 is provided with two second pins 221, and the second pins 221 are electrically connected to the circuit board 22. Second through holes 2114 are provided at the bottom of the bottom case 211, second pins 221 extend from the circuit board 22 into the second through holes 2114, and each of the second pins 221 has a jack. When charging, the first pins 13 are inserted in the jacks to charge the battery 23. It should be understood that, in other embodiments of the present application, a socket can also be disposed on the handle 2, and then a plug is disposed on the charging base 1, so that, when charging, the plug is inserted into the socket to form an electric connection.

Further, referring to FIGS. 4-7, as a specific embodiment of the electric toothbrush provided by the present application, the charging base 1 is provided with a first magnetic member 14. The enclosure 21 (specifically, the bottom case 211) is provided with a second magnetic member 2112. The first magnetic member 14 and the second magnetic member 2112 are both magnets, and the first magnetic member 14 and the second magnetic member 2112 are attracted to each other so that the first pins 13 are inserted into the jacks. During charging, users only need to align the first pins 13 with the jacks, and then insert the first pins 13 into the jacks through the suction of the first magnetic member 14 and the second magnetic member 2112, and maintain the electric connection state of the first pins 13 and the second pins 221.

Preferably, in this embodiment, the charging base 1 is provided with two first magnetic members 14, and the two first magnetic members 14 are symmetrically disposed on opposite two sides of the two first pins 13, respectively, and corresponding, the bottom case 211 is provided with two second magnetic members 2112, and the two second magnetic members 2112 are symmetrically disposed on opposite two sides of the two second pins 221, respectively, so that good electrical connection between the two first pins 13 and the two second pins 221 can be fully guaranteed.

Further, the bottom of the bottom case 211 is provided with first magnetic slots 2113, and the first magnetic members 14 are disposed in the first magnetic slots 2113. The top inner side of the charging base 1 is provided with second magnetic slots 15, and the second magnetic members 2112 are disposed in the second magnetic slots 15.

The above are only the preferred embodiments of the present application and are not intended to limit the present application. Any modification, equivalent replacement, and improvement made within the spirit and principle of the present application shall be included in the protection scope of the present application.

1. An electric toothbrush comprising:
a handle, a toothbrush head, and a charging base, wherein
one end of the handle is configured for outputting ultrasonic vibrations, the toothbrush head is configured for simultaneously wrapping upper and lower rows of teeth of a human body, and the toothbrush head is configured to be disposed on the end of the handle and vibrates with the end of the handle to simultaneously clean the upper and lower rows of teeth of the human body; and the charging base is disposed at the other end of the handle and charges the handle, and the charging base comprises a sterilization unit for sterilizing the handle and the toothbrush head.

2. The electric toothbrush according to claim 1, wherein the toothbrush head comprises:
a bristle holder, in a tooth shape adapted to a shape of the upper and lower rows of teeth of the human body, and the bristle holder has a first surface and a second surface opposite to each other;
first bristles, disposed on the first surface and configured for wrapping and cleaning the upper row of teeth of the human body; and
second bristles, disposed on the second surface and configured for wrapping and cleaning the lower row of teeth of the human body.

3. The electric toothbrush according to claim 2, wherein the toothbrush head further comprises a transmitting frame, and the transmitting frame comprises:
a transmitting body, inserted in the bristle holder and configured for forming a connection with the end of the handle; and
a plurality of transmitting rods, distributed on the transmitting body and configured for dispersing the ultrasonic vibrations on the transmitting body to various parts of the bristle holder.

4. The electric toothbrush according to claim 3, wherein the bristle holder comprises a middle portion, and a first side portion and a second side portion respectively disposed at both ends of the middle portion; the first side portion, the middle portion and the second side portion are sequentially connected to form a tooth-shaped bristle holder; and the transmitting body is disposed on the middle portion, a part of the transmitting rods are disposed on the first side portion and are spaced along the width direction of the first side portion, and another part of the transmitting rods are disposed on the second side portion and are spaced along the width direction of the second side portion.

5. The electric toothbrush according to claim 3, wherein the handle comprises:
an enclosure;
a circuit board, disposed in the enclosure;
a battery, disposed in the enclosure and configured for supplying power to the circuit board; and
a vibration motor, disposed in the enclosure and electrically connected to the circuit board, wherein a motor shaft of the vibration motor passes through the enclosure to be connected to the transmitting body.

6. The electric toothbrush according to claim 5, wherein the enclosure comprises a bottom case and an upper cover, and the bottom case is engaged with the upper cover and sealed by a sealing ring; a first through hole is provided at a junction of the bottom case and the upper cover, and a button configured for driving the vibration motor is disposed in the first through hole; and the sealing ring is integrally connected to the button.

7. The electric toothbrush according to claim 5, wherein the handle further comprises blue light lamps electrically connected to the circuit board and capable of emitting blue light to whiten teeth, the blue light emitted by the blue light lamp shines on the bristle holder through the enclosure, and the bristle holder is made of a light guide material.

8. The electric toothbrush according to claim 7, wherein the handle comprises two blue light lamps, and the two blue light lamps are symmetrically disposed on opposite two sides of the motor shaft, respectively.

9. The electric toothbrush according to claim 5, wherein the charging base further comprises a drying unit configured for drying the handle and the toothbrush head; and the charging base has two first pins, the handle is provided with two second pins electrically connected to the circuit board, the second pins have jacks, and the first pins are inserted into the jacks to charge the battery.

10. The electric toothbrush according to claim 9, wherein a first magnetic member is disposed on the charging base, a second magnetic member is provided on the enclosure, and the first magnetic member and the second magnetic member are attracted to each other, so that the first pins are inserted into the jacks.
